(19) United States
(12) Patent Application Publication
(10) Pub. No.: US 2026/0216432 A1
(43) Pub. Date: Jul. 30, 2026

(54) Medicament delivery device

(71) Applicant: SHL Medical AG, Zug (CH)

(72) Inventor: Nurettin Ali, Deerfield Beach, FL (US)

(21) Appl. No.: 19/143,685

(22) PCT Filed: Dec. 22, 2023
(86) PCT No.: PCT/EP2023/087578

§ 371 Date: Jun. 26, 2025

Related U.S. Application Data
(60) Provisional application No. 63/436,987, filed on Jan. 4, 2023.

(30) Foreign Application Priority Data
Feb. 2, 2023 (EP) 23154595.5

(51) Int. Cl. A61M 5/20

(52) U.S. Cl. CPC A61M 5/20; A61M 2005/208

(57) ABSTRACT

The present disclosure provides a medicament delivery device having a proximal end pointing towards a dose delivery site during use of the medicament delivery device and a distal end opposite the proximal end. The medicament delivery device includes a housing having a longitudinal axis. The medicament delivery device further includes a plunger rod comprising a plunger rod boss and being rotatable between a first rotational position and a second rotational position, wherein the plunger rod is axially fixed with respect to the housing in the first rotational position and axially moveable with respect to the housing in the second rotational position. The medicament delivery device further includes a medicament delivery member cover coupled to the housing and movable between an extended position and a retracted position. The housing comprises a housing sloped surface and a housing slot recessed on an inner circumferential surface of the housing. In the first rotational position, the plunger rod boss abuts the sloped surface, and the sloped surface is configured to block a proximal movement of the plunger rod boss towards the medicament delivery site. The medicament delivery member cover comprises at least one medicament delivery member cover arm extending in a distal direction, wherein the distal direction is pointing away from a medicament delivery site. In the extended position, the plunger rod boss abuts a distal end of the medicament delivery member cover arm, wherein the distal end is configured to block rotation of the plunger rod boss.

### TECHNICAL FIELD

The present disclosure relates to a medicament delivery device and in particular to an activation mechanism of a medicament delivery device.

### BACKGROUND

A medicament delivery device, in particular an autoinjector, according to the prior art comprises a housing comprising a front housing and a rear housing. The housing is adapted to hold a medicament container, such as a syringe. The syringe may be a pre-filled syringe and have a needle as a medicament delivery member arranged at a proximal end. When the autoinjector and/or the syringe are assembled, a protective needle sheath may be removably coupled to the needle. The protective needle sheath may be made of a flexible shell (for instance composed of rubber) and/or a rigid shell (for instance composed of plastic). A stopper, also called plunger, may be arranged for sealing the syringe distally and for expelling a medicament contained in the syringe through the needle. In other exemplary embodiments, the medicament container may be a cartridge which includes the medicament and engages a removable needle (e.g., by threads, snaps, friction, etc.).

A cap may be removably disposed at a proximal end of the housing. The cap may include an element (e.g., a barb, a hook, a narrowed section, etc.) arranged to engage the protective needle sheath, the housing and/or a medicament delivery member cover, also referred to as needle cover, telescoped within the housing. The cap may comprise grip features for facilitating removal of the cap (e.g., by twisting and/or pulling the cap relative to the housing).

A medicament delivery member cover biasing member (for instance a coil spring) may be arranged to bias the medicament delivery member cover in a proximal direction relative to the housing, before injection, for safety reasons. The medicament delivery member cover biasing member needs to be compressed at the beginning of injection in order to retract the medicament delivery member cover inside the housing and uncover the medicament delivery member, thereby activating the device.

A pre-loaded drive biasing member, for instance a pre-compressed drive coil spring, may be arranged within the housing. A plunger rod may be used to forward force from the drive biasing member to the stopper. In an exemplary embodiment, the plunger rod is hollow and the drive biasing member is arranged within the plunger rod. In another exemplary embodiment, the plunger rod is solid and the drive biasing member engages a distal end of the plunger rod. When the pre-loaded drive biasing member is released, the drive biasing member biases the plunger rod in the proximal direction relative to the housing, releasing the plunger rod. Besides that, the plunger rod may itself comprise means for sealing the medicament container such that no stopper needs to be provided.

A plunger rod release mechanism may be arranged for preventing release of the plunger rod prior to retraction of the medicament delivery member cover relative to the housing and for releasing the plunger rod only once the medicament delivery member cover is sufficiently retracted.

A medicament delivery member cover lock mechanism (needle cover lock mechanism) may be arranged to prevent retraction of the medicament delivery member cover relative to the housing when the cap is in place, thereby avoiding unintentional activation of the autoinjector (e.g., if dropped, during shipping or packaging, etc.). Such mechanisms are known in the art and will not be described in detail.

However, there is a need to further improve the triggering mechanism of medicament delivery devices while effectively preventing inadvertent activation of the device.

This need is met by a device according to the present disclosure.

### SUMMARY

Reference should now be made to the appended claims.

The invention relates to a medicament delivery device, the medicament delivery device having a proximal end pointing towards a dose delivery site during use of the medicament delivery device, and a distal end opposite the proximal end, the medicament delivery device comprising:
a housing having a longitudinal axis,
a plunger rod comprising a plunger rod boss and being rotatable between a first rotational position and a second rotational position, wherein the plunger rod is axially fixed with respect to the housing in the first rotational position and axially moveable with respect to the housing in the second rotational position,
a medicament delivery member cover coupled to the housing and movable between an extended position and a retracted position,
wherein the housing comprises a housing sloped surface and a housing slot recessed on an inner circumferential surface of the housing,
wherein in the first rotational position, the plunger rod boss abuts the sloped surface, and the sloped surface is configured to block a proximal movement of the plunger rod boss towards the medicament delivery site,
wherein the medicament delivery member cover comprises at least one medicament delivery member cover arm extending in a distal direction, wherein the distal direction is pointing away from a medicament delivery site, and
wherein in the extended position, the plunger rod boss abuts a distal end of the medicament delivery member cover arm, wherein the distal end is configured to block rotation of the plunger rod boss.

In a non-limiting embodiment, when the medicament delivery member cover is pushed in the distal direction into the retracted position, the distal end disengages the plunger rod boss and the plunger rod boss is rotated into the second rotational position, and in the second rotational position, the plunger rod boss is axially moveable in the housing slot in the proximal direction.

In a non-limiting embodiment, the distal end is a distal circumferential surface of the medicament delivery member cover arm.

In a non-limiting embodiment, the sloped surface is configured to guide the rotation of the plunger rod boss and thus the plunger rod from the first rotational position to the second rotational position.

In a non-limiting embodiment, the medicament delivery member cover arm further comprises an axial slot recessed from the distal end in a circumferential direction and extending in the proximal axial direction.

In a non-limiting embodiment, the housing slot extends in the axial direction and the axial slot and the housing slot are aligned in the axial direction, and in the second rotational position, the plunger rod boss is axially moveable in the axial slot.

In a non-limiting embodiment, the sloped surface and the housing slot are connected.

In a non-limiting embodiment, the medicament delivery device further comprises a rear housing provided at a distal end of the housing.

In a non-limiting embodiment, the rear housing comprises a rear housing pocket, and the plunger rod boss is positioned in the rear housing pocket (24) in first rotational position.

In a non-limiting embodiment, the rear housing comprises a rear housing slot being connected to the rear housing pocket and extending in the proximal direction, and in the second rotational position, the plunger rod boss is positioned in the rear housing slot.

In a non-limiting embodiment, the rear housing comprises at least one snap fit provided towards a distal end of the rear housing and the housing comprises a corresponding housing recess, and during assembly, the snap fit is configured to engage the housing recess to couple the rear housing to the housing.

The invention is specified by the independent claim. Preferred embodiments are defined in the dependent claims. In the following description, although numerous features may be designated as optional, it is nevertheless acknowledged that all features comprised in the independent claims are not to be read as optional.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a longitudinal axis extending in the direction of the longest extension of the device and/or component. Similarly, “radial” or “radially” refer to a direction extending radially relative to the axis, and “rotation”, “rotational” and “rotationally” refer to rotation relative to the axis.

When a component is said to move proximally, distally, axially in a proximal direction, axially in a distal direction or equivalent terms, the movement is relative to the housing of the injection device, unless mentioned otherwise.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1A shows a partial view of a rear housing according to an embodiment of the disclosure.

FIG. 1B shows a partial view of a plunger rod according to an embodiment of the disclosure.

FIG. 1C shows the plunger rod of FIG. 1B assembled to the rear housing of FIG. 1A.

FIG. 1D shows a partial view of a housing according to an embodiment of the disclosure.

FIG. 1E shows a medicament delivery member cover according to an embodiment of the disclosure.

FIG. 2A shows a partial view of a medicament delivery device according to an embodiment of the disclosure in an assembled state.

FIG. 2B shows the medicament delivery device of FIG. 2A in an activated state.

FIGS. 3A and 3B show the medicament delivery device of FIGS. 2A and 2B, respectively.

FIGS. 4A to 4C show elements of a medicament delivery device according to the present disclosure during assembly.

FIG. 5 is an exploded view of a medicament delivery device according to an embodiment of the present disclosure.

### DETAILED DESCRIPTION

The activation mechanism according to the present invention can be employed for various medicament delivery devices. Thus, only the parts relevant for carrying out the disclosure will be described in detail.

In the figures, elements not essential for carrying out the invention have been omitted. In particular, not all figures show a complete outer housing, biasing members, a medicament container, cap, etc. in order not to render the depictions overly complicated and not to blur the scope of the disclosure.

In the following, the assembled state refers to a state in which the device is delivered to the patient, i.e., before activation and medicament delivery. In the assembled state, the plunger rod is in a locked position and not able to rotate or move in the proximal direction. In the activation state, the medicament delivery member cover is moved in the distal direction along the longitudinal axis from an extended position to a retracted position. In the activated state, the plunger rod is free to rotate and move in the proximal direction for medicament delivery. The plunger rod may thus be displaceable along the axial direction.

The medicament delivery member cover can be moved from the extended to the retracted position. Therefore, the medicament delivery member cover can be displaced in the axial direction. In the extended position, the medicament delivery member cover extends in a proximal direction from the housing. Thereby, a medicament delivery member, e.g., a needle, may be covered by the medicament delivery member cover. By pushing the medicament delivery member cover in the distal direction, e.g., by pushing the device onto a medicament delivery site, the medicament delivery member cover is moved to the retracted position for activating medicament delivery. After delivery, the medicament delivery member cover may be moved back to the extended position to cover the medicament delivery member. The medicament delivery member cover may also be locked in the extended position after medicament delivery to and prevent injuries of the patient.

In the following, the term engagement encompasses any kind of interaction between the named features such as abutment, clamping, locking, biasing, flexing, etc.

The embodiments described below are examples of the present disclosure and may be combined unless indicated otherwise.

The present disclosure employs a rotating plunger rod configuration.

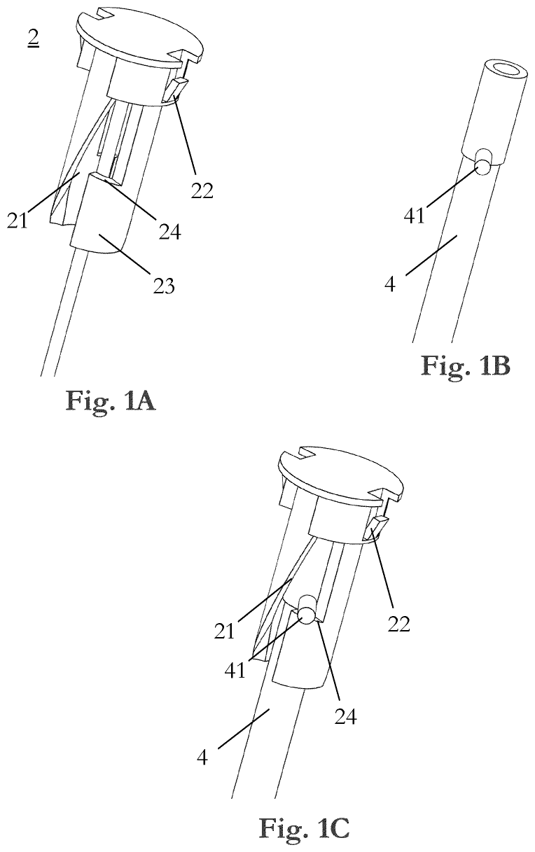

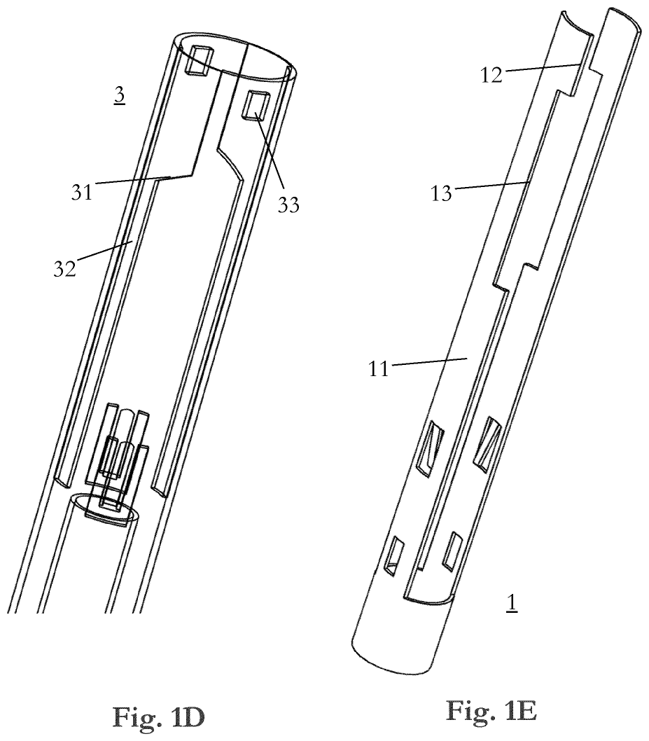

FIG. 1A is a partial perspective view of a rear housing 2 according to an embodiment. The rear housing may comprise at least two rear housing arms 23 which are separated in the circumferential direction by at least two rear housing slots 21. The slots 21 may substantially extend in the axial direction and may be open towards the proximal end of the rear housing 2. The rear housing 2 may further comprise at least two rear housing pockets 24 provided in respective rear housing arms 23 and connected to respective slots 21. The rear housing pockets 24 may be recessed in the circumferential direction with regard to the respective slot 21 and may have a proximal abutment surface configured to receive a plunger rod boss 41. The proximal abutment surface may be sloped in the proximal direction in order to secure the plunger rod boss 41. The slots 21 and the pockets 24 may be formed as cut-outs.

The rear housing 2 may further comprise at least two snap fits 22 provided at or towards a distal end of the rear housing 2 and extending in the radial outward direction.

The rear housing may also comprise a central rod configured to receive a drive biasing member 6 (not shown), for instance a coil spring.

FIG. 1B shows a plunger rod 4 according to an embodiment of the disclosure. The plunger rod 4 comprises at least two plunger rod bosses 41 extending in a radial outward direction. The plunger rod bosses are located to point in opposite directions (FIG. 1B shows only one of the two bosses 41). The plunger rod bosses 41 may be located towards the distal end of the plunger rod 4. However, the location of the plunger rod bosses 41 may be changed and other corresponding elements of the device may be adapted accordingly.

FIG. 1C shows the plunger rod 4 of FIG. 1B assembled to the rear housing 2 of FIG. 1A. The plunger rod boss 41 is shown in a configuration during assembly, i.e., prior to the assembled state, and abuts a corresponding proximal abutment surface of the pocket 24.

The plunger rod 4 may be pre-tensioned in the axial direction by a drive biasing member 6 (not shown), which can be any type of energy accumulating member, for instance a coil spring, which urges the plunger rod 4 in the proximal direction. In FIG. 1C, the plunger rod 4 is locked in the proximal direction due to the abutment of the plunger rod boss 41 on the pocket 24, in particular the proximal abutment surface.

FIG. 1D shows a partial perspective view of a housing 3 according to an embodiment. The housing 3 may be generally tubular, i.e., substantially cylindrical. The housing 3 comprises a housing sloped surface 31 and a housing slot 32. The housing sloped surface 31 and the housing slot 32 may be recessed on an inner circumferential surface of the housing 3. The housing slot 32 may extend in the axial direction and may be connected to the housing sloped surface 31. The housing sloped surface 31 may be sloped in the proximal direction towards the housing slot 32.

FIG. 1E shows a medicament delivery member cover 1 according to an embodiment. The medicament delivery member cover 1 may comprise at least two arms 11 extending in the distal direction. The medicament delivery member cover arms 11 may each comprise a distal end 12. Moreover, the medicament delivery member cover arms 11 may comprise an axial slot 13 recessed in the circumferential direction. The axial slot 13 may extend from the distal end 12 in the proximal direction.

The present disclosure is exemplarily described with two plunger rod bosses 41. However, other numbers, e.g. one, three or four, may also be used without compromising the functionality. In general, the number of rear housing slots 21, rear housing pockets 24, housing sloped surfaces 31, housing slots 32, and medicament delivery member arms 11 may correspond to the number of plunger rod bosses 41.

The medicament delivery member cover 1 may be located radially inside the housing 3, the rear housing arms 23 may be located radially inside the medicament delivery member cover 1 and the plunger rod 4 may be located inside the rear housing arms 23. The plunger rod boss 41 may extend through respective recesses or cut-outs of the rear housing 2 and the medicament delivery member cover 1 and may be in engagement with the housing sloped surface 31 and the housing slot 32.

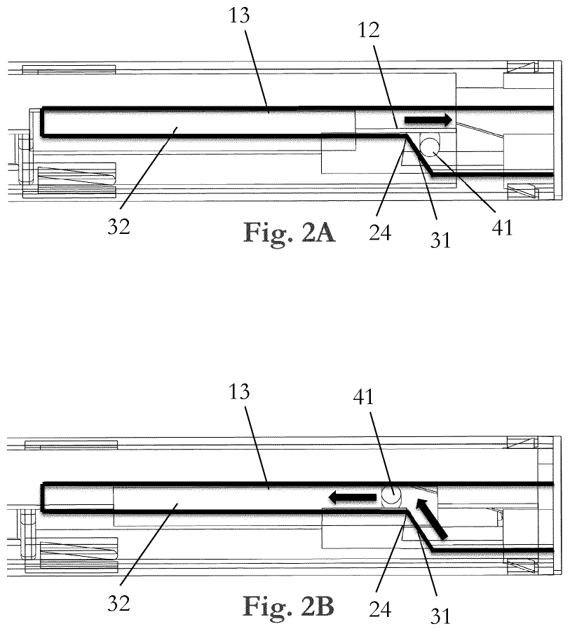

FIG. 2A shows a partial view of a medicament delivery device according to an embodiment in the assembled state, i.e., prior to activation. The housing 3 is transparently displayed, the housing sloped surface 31 and the housing slot 32 have been highlighted by a thick line. The medicament delivery member cover 1 is in its extended position, the plunger rod 4 is in the first rotational position. The medicament delivery device comprises the elements of FIGS. 1A to 1E.

In the assembled state, the plunger rod boss 41 abuts the sloped surface 31 of the housing 3 in the proximal direction and the distal end 12 of the medicament delivery member 1 in the circumferential direction. In particular, the plunger rod boss 14 may abut a circumferentially oriented lateral side of the distal end 12. Movement of the plunger rod boss 41, and therefore the plunger rod 4, is blocked in the axial direction by the sloped surface 31 and in the circumferential (rotational) direction by the distal end 12. The plunger rod boss 41 may be positioned in the rear housing pocket 24.

In the activation state, the medicament delivery member cover 1 is pushed in the distal direction, e.g., by pushing the medicament delivery device on a medicament delivery site. This is indicated by the arrow in FIG. 2A.

FIG. 2B shows the device of FIG. 2A in the activated state. The medicament delivery member cover 1 has been pushed in the distal direction such that the plunger rod boss 41 no longer abuts the distal end 12. The plunger rod boss 41 is thus free to rotate and slides along the sloped surface 31 to rotate the plunger rod 4 from the first rotational position to the second rotational position. In the second rotational position, the plunger rod boss 41 is axially moveable in the housing slot 32 and pushed in the proximal direction by the drive biasing member 6 to expel the medicament (as indicated by the arrows). Moreover, the plunger rod boss 41 is located in and slides along the axial slot 13 which is aligned with the housing slot 32. In the second rotational position, the plunger rod 41 is also located in the rear housing slot 21 allowing the plunger rod boss 41 to move in the proximal direction.

In other words, when the medicament delivery member cover 1 is pushed a certain distance in the distal direction, the abutment of the plunger rod boss 41 on the distal end 12 is released and the plunger rod boss 41 may enter the axial slot 13, thus allowing the plunger rod 4 to rotate. The sloped surface 31 is sloped to guide and/or aid the rotational movement of the plunger rod 4. In particular, the sloped surface 31 may be sloped in the proximal direction towards the housing slot 32 such that the plunger rod boss 41 slides along the sloped surface 31 due to the force of the drive biasing member 6, thus rotating the plunger rod until the housing slot 32 is reached and the plunger rod boss 41 may be moved in the axial proximal direction. Due to the sloped surface 31, the rotation from the first rotational position to the second rotational position is a combination of rotational movement and axial proximal movement.

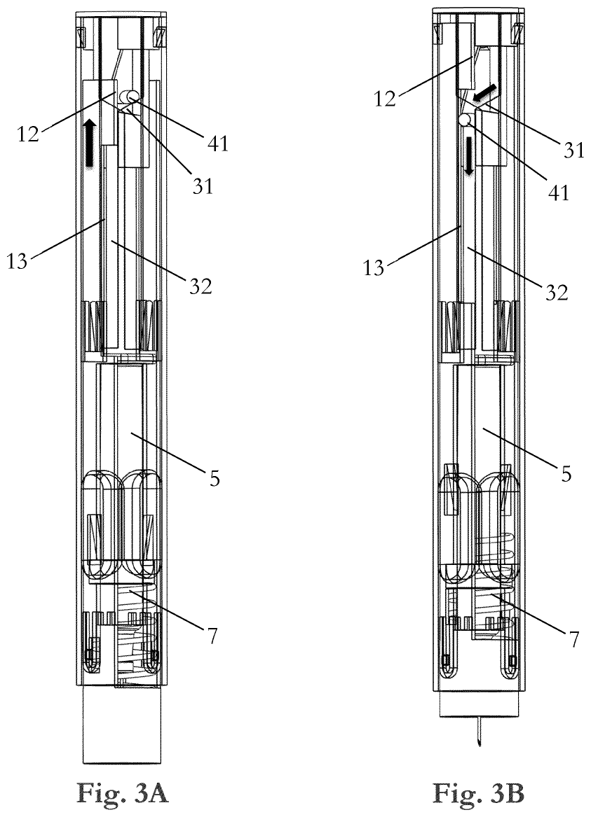

FIGS. 3A and 3B are views of a medicament delivery device corresponding to FIGS. 2A and 2B, respectively.

The medicament delivery device comprises a housing 3 having a longitudinal axis, a plunger rod 4 comprising a plunger rod boss 41 and being rotatable between a first rotational position and a second rotational position, wherein the plunger rod 4 is axially fixed with respect to the housing 3 in the first rotational position and axially moveable with respect to the housing 3 in the second rotational position. A medicament delivery member cover 1 is coupled to the housing 3 and is movable between an extended position and a retracted position. The housing 3 comprises a housing sloped surface 31 and a housing slot 32 recessed on an inner circumferential surface of the housing 3.

In the first rotational position, the plunger rod boss 41 abuts the sloped surface 31 and the sloped surface 31 is configured to block a proximal movement of the plunger rod 4 towards the medicament delivery site.

The medicament delivery member cover 1 comprises at least one medicament delivery member cover arm 11 extending in a distal direction, wherein in the extended position, the plunger rod boss 41 abuts a distal end 12 of the medicament delivery member cover arm 11, the distal end 12 being configured to block rotation of the plunger rod. When the medicament delivery member cover 1 is pushed in the distal direction into the retracted position, the distal end 12 disengages the plunger rod boss 41 and the plunger rod 4 is rotated into the second rotational position. In the second rotational position, the plunger rod boss 41 is axially moveable in the housing slot 32 in the proximal direction.

The distal end 12 may be a distal circumferential surface of the medicament delivery member cover arm 11. That is, the distal end 12 may be a lateral side surface of a distal end of the medicament delivery member cover 1 and medicament delivery member cover arm 11, respectively. The sloped surface 31 may be configured to guide the rotation of the plunger rod boss 41 and thus the plunger rod 4 from the first rotational position to the second rotational position.

The medicament delivery member cover arm 11 may further comprise an axial slot 13 recessed from the distal end 12 in a circumferential direction and extending in the proximal axial direction. The housing slot 32 may extend in the axial direction. The axial slot 13 and the housing slot 32 may be aligned in the axial direction. In the second rotational position, the plunger rod boss 41 may be axially moveable in the axial slot 13. The sloped surface 31 and the housing slot 32 may be connected.

The medicament delivery device may further comprise a rear housing 2 as described above provided at a distal end of the housing 3. In the first rotational position, the plunger rod boss 41 may be positioned in the rear housing pocket 24. In the second rotational position, the plunger rod boss 41 may be positioned in the rear housing slot.

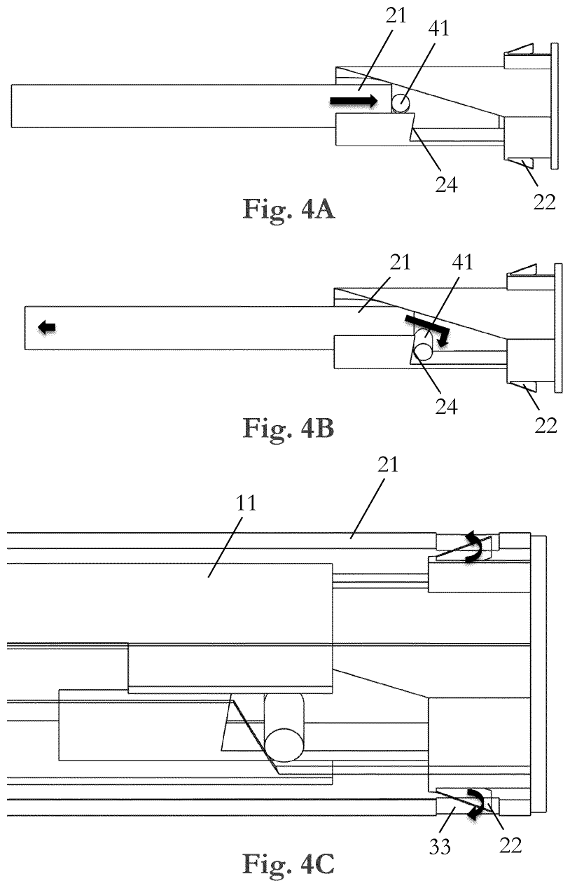

Assembly of a medicament delivery device according to the above will now briefly described with reference to FIGS. 4A to 4C.

As shown in FIG. 4A, the plunger rod 4 is pushed in the distal direction onto the rear housing 2 against the force of the drive biasing member 6 (not shown, located inside the substantially hollow cylindrical plunger rod). In particular, the plunger rod 4 is pushed such that the plunger rod boss 41 is entered through the rear housing slot 21.

With reference to FIG. 4B, when the rear housing pocket 24 is reached, the plunger rod 4 is rotated such that the plunger rod boss 41 engages the plunger rod pocket 24 and abuts the proximal abutment surface of the pocket 24. The plunger rod boss 41 is pushed against the proximal abutment surface by the force of the drive biasing member 6. A slope in the proximal direction may ensure that the plunger rod boss 41 is not rotated out of the pocket 24 in the shown assembly state and the plunger rod 4 may thus slightly move in the proximal direction as indicated by the arrows. The power pack consisting of the plunger rod 4, the rear housing 2 and the drive biasing member 6 is thus assembled.

FIG. 4C shows the medicament delivery device after assembly of the power pack to the housing 3.

As described above, the rear housing 2 may comprise at least one snap fit 22 provided towards a distal end of the rear housing 2 and the housing 3 may comprise a corresponding housing recess 33. The recess 33 may also be formed as a cut-out as shown in the figures.

The power pack of rear housing 2, drive biasing member 6 and plunger rod 4 is entered into the housing 3 from the distal end of the housing 3.

During assembly, the snap fit 22 is configured to engage the housing recess 33 to couple the rear housing 2 to the housing 3.

The snap fit 22 may be formed as a flexible arm, snap, latch or the like which is pushed radially inward during assembly due to the abutment with the inner circumferential surface of the housing 3 and flexes back outwards when the recess 33 is reached. The snap fit 22 may abut a distal end surface of the recess to secure the power pack in the device. The device is in the assembled state.

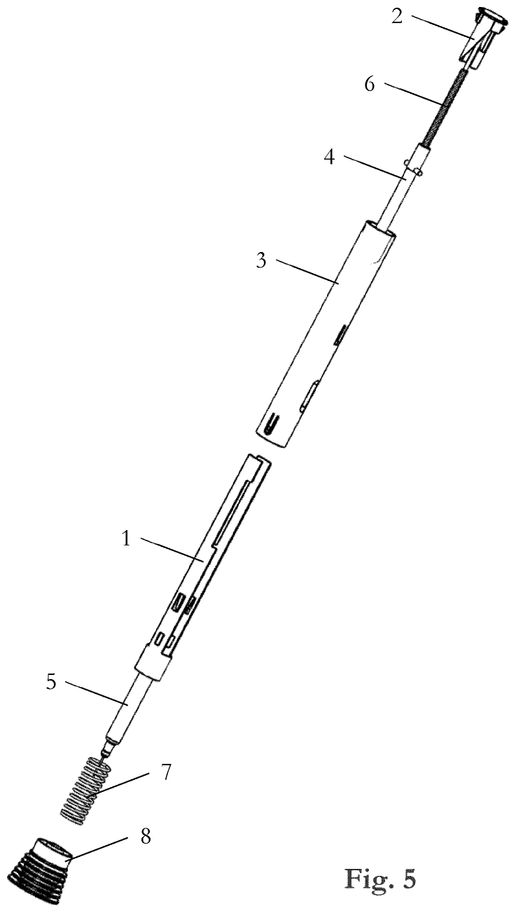

FIG. 5 shows an exploded view of a medicament delivery device as described above comprising a cap 8, a medicament delivery member cover biasing member 7 (for instance a coil spring), a syringe 5, a medicament delivery member cover 1, a housing 3, a plunger rod 4, a drive biasing member 6 and a rear housing 2.

In the present disclosure, improved mechanisms for activation of a medicament delivery device and in particular for release of a plunger rod have been presented. The present disclosure provides a simplified plunger rod design having only one feature, i.e., pin, that is used for installation and activation. This leads to an overall reduced complexity of the device.

Also, according to the present disclosure, handling of the medicament delivery device may be facilitated while preventing inadvertent activation of the device and injuries of a user.

Moreover, an improved assembly is provided by the present disclosure.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

### REFERENCE SIGNS

| | |
|---|---|
| 1 | Medicament delivery member cover/needle cover |
| 11 | Medicament delivery member cover arm/needle cover arm |
| 12 | Distal end |
| 13 | Axial slot |
| 2 | Rear housing/rear cap |
| 21 | Rear housing slot |
| 22 | Snap fit |
| 23 | Rear housing arms |
| 24 | Rear housing pocket |
| 3 | Housing |
| 31 | Housing sloped surface |
| 32 | Housing slot |
| 33 | Housing recess/cut-out |
| 4 | (Rotatable) Plunger rod |
| 41 | Plunger rod boss |
| 5 | Syringe |
| 6 | Drive biasing member |
| 7 | Medicament delivery member cover biasing member/needle |
| | cover biasing member |
| 8 | Cap |

1-11. (canceled)

12. A medicament delivery device, the medicament delivery device having a proximal end pointing towards a dose delivery site during use of the medicament delivery device, and a distal end opposite the proximal end, the medicament delivery device comprising:
a housing having a longitudinal axis;
a plunger rod comprising a plunger rod boss and being rotatable between a first rotational position and a second rotational position, wherein the plunger rod is axially fixed with respect to the housing in the first rotational position and axially moveable with respect to the housing in the second rotational position; and
a medicament delivery member cover coupled to the housing and movable between an extended position and a retracted position,
wherein the housing comprises a housing sloped surface and a housing slot recessed on an inner circumferential surface of the housing,
wherein in the first rotational position, the plunger rod boss abuts the sloped surface, and the sloped surface is configured to block a proximal movement of the plunger rod boss towards the medicament delivery site,
wherein the medicament delivery member cover comprises at least one medicament delivery member cover arm extending in a distal direction, wherein the distal direction is pointing away from a medicament delivery site, and
wherein in the extended position, the plunger rod boss abuts a distal end of the medicament delivery member cover arm, wherein the distal end is configured to block rotation of the plunger rod boss.

13. The medicament delivery device according to claim 12, wherein, when the medicament delivery member cover is pushed in the distal direction into the retracted position, the distal end disengages the plunger rod boss and the plunger rod boss is rotated into the second rotational position, and
wherein in the second rotational position, the plunger rod boss is axially moveable in the housing slot in the proximal direction.

14. The medicament delivery device according to claim 12, wherein the distal end is a distal circumferential surface of the medicament delivery member cover arm.

15. The medicament delivery device according to claim 12, wherein the sloped surface is configured to guide the rotation of the plunger rod boss and thus the plunger rod from the first rotational position to the second rotational position.

16. The medicament delivery device according to claim 12, wherein the medicament delivery member cover arm further comprises an axial slot recessed from the distal end in a circumferential direction and extending in the proximal axial direction.

17. The medicament delivery device according to claim 16, wherein the housing slot extends in the axial direction and the axial slot and the housing slot are aligned in the axial direction,
wherein in the second rotational position, the plunger rod boss is axially moveable in the axial slot.

18. The medicament delivery device according to claim 12, wherein the sloped surface and the housing slot are connected.

19. The medicament delivery device according to claim 12, wherein the medicament delivery device further comprises a rear housing provided at a distal end of the housing.

20. The medicament delivery device according to claim 19, wherein the rear housing comprises a rear housing pocket, wherein the plunger rod boss is positioned in the rear housing pocket in first rotational position.

21. The medicament delivery device according to claim 20, wherein the rear housing comprises a rear housing slot being connected to the rear housing pocket and extending in the proximal direction,
wherein in the second rotational position, the plunger rod boss is positioned in the rear housing slot.

22. The medicament delivery device according to claim 19, wherein the rear housing comprises at least one snap fit provided towards a distal end of the rear housing and the housing comprises a corresponding housing recess,
wherein during assembly, the snap fit is configured to engage the housing recess to couple the rear housing to the housing.
